(19) United States
(12) Patent Application Publication
(10) Pub. No.: US 2025/0034546 A1
(43) Pub. Date: Jan. 30, 2025

(54) Deblocking chromatography and cassette for isolating nucleic acids from liquid biopsy samples

(71) Applicants: Shaofeng Ding, Santa Fe Springs, CA (US); Qiang Liu, Rancho Cucamonga, CA (US)

(72) Inventors: Shaofeng Ding, Santa Fe Springs, CA (US); Qiang Liu, Rancho Cucamonga, CA (US)

(21) Appl. No.: 18/771,811

(22) Filed: Jul. 12, 2024

Related U.S. Application Data
(60) Provisional application No. 63/528,647, filed on Jul. 25, 2023.

(51) Int. Cl. C12N 15/10

(52) U.S. Cl. CPC C12N 15/101

(57) ABSTRACT

The instant invention provides a deblocking double-layer chromatography and cassette for isolating nucleic acids from liquid biopsy samples such as urine with large sample volumes, small nucleic acid sizes, and low nucleic acid concentrations. Compared with the prior art of Ding et al. (U.S. Pat. No. 9,163,230) which proved the principle of double-layer chromatography comprising a positively charged DEAE membrane and a silica membrane, we further explored and optimized the membrane compositions and solution compositions for plasma and urine samples. More importantly, we set up a “deblocking” mechanism to overcome a “blocking” problem that the membrane is blocked by solid particles in the plasma and urine samples, thus greatly increasing its flowability. In addition, it omits a pre-filtering step, thus particularly suitable for its automation.

### SEQUENCE LISTING

This instant application contains a Sequence Listing which is submitted in XML format via Patent Center and is hereby incorporated by reference in its entirety. Said XML file is named “sequencelisting_st26” was created on Feb. 29, 2024 and is 6000 bites in size.

### BACKGROUND

The present invention discloses a deblocking chromatography and cassette for isolating nucleic acids particularly from liquid biopsy samples such as urine with large sample volumes, small nucleic acid sizes and low nucleic acid concentrations.

### Nucleic Acid Isolation

Nucleic acid isolation is an important step for many biochemical and diagnostic uses, e.g., cloning, transformation, restriction digestion, in vitro transcription, amplification, and sequencing. However, it can not be easily carried out because of the presence of large amounts of cellular and other contaminants, e.g., proteins, carbohydrates and small metabolites in crude samples. Thus, simple, efficient, reliable, and automatic methods are needed.

### Silica-Based Isolation

Silica or silicon dioxide is hydrophilic due to silanol (Si—OH) residues on the surface which interact strongly with polar water molecules.

The silica- or silicon dioxide-based method has become a popular method for isolating nucleic acids that use controlled pore glass, filters embedded with silica particles, silica gel particles, resins comprising silica in the form of diatomaceous earth, and glass fibers [1-9].

The basic procedure is simple. DNA or RNA is bound to the surface of silica resin or membrane in the presence of a high concentration of chaotrophic salts, contaminants are washed away, and DNA or RNA is eluted in water or low-salt buffer.

The principle of silica-based isolation is based on the high affinity of the negatively charged DNA/RNA backbone towards the positively charged silica surface under concentrated chaotrophic salt conditions, e.g., sodium iodide (NaI), sodium perchlorate (NaClO₄), guanidinium hydrochloride (GuHCl), and guanidinium thiocyanate (GuTC).

The effects of ionic strength, temperature, pH, DNA size and conformation on the binding of nucleic acids to the silica surface were investigated [11]. For example, the binding capacity of the silica surface is linearly related to the chaotrophic salt concentration. Additionally, at a given chaotrophic salt concentration, the binding capacity to the silica surface of any type of DNA/RNA is higher at a lower pH.

GuTC and GuHCl are commonly used for binding nucleic acids to the silica surface. NaI and NaClO₄ are also used to a lesser extent. GuTC at a concentration of 4 M to 6 M works best, while GuHCl is used at a higher concentration of up to 6 M. The binding efficiency is significantly improved in the presence of ethanol or propanol. In order to control the pH of the binding reagent, sodium acetate and Tris-HCl buffers, ranging from pH 6 to 7.5 are often used. Moreover, guanidinium salts, e.g., GuTC, are known to efficiently lyse cells and denature proteins [4], eliminating the need for adding denaturing enzymes, e.g., proteinase.

Spin and vacuum chromatography columns are commonly used within which silica resin or membrane is packed as the stationary phase, e.g., those of Pure Yield plasmid midiprep system and WizardPlus minipreps DNA purification system from Promega, QIAprep miniprep kit from Qiagen, and EZgene plasmid purification miniprep kit from Bioland.

The silica-based method provides a quick, convenient, and efficient method. The purified DNA is qualified and ready to use for a wide variety of downstream applications. However, the starting volume of samples, e.g., plasma, is limited because up to 3 volumes of chaotrophic salt solution should be added to one volume of the DNA or RNA samples in order to reach the effective chaotropic salt concentration required by silica binding. Thus, it is not suitable for liquid biopsy samples such as urine with large sample volumes, low nucleic acid concentrations and small nucleic acid sizes.

### Other Types of Membrane or Bead-Based Isolation Besides Silica

Silicon carbide, another silicate compound, when oxygen links to the surface, tends to behave closer to the properties of silica. Silicon carbide binds to DNA or RNA and then dissociates them under a different condition, which is used in a spin column in Norgen urine cell-free circulating DNA purification kit.

Cellulose membrane or bead, such as Waterman cellulose filter papers No. 1, 3 and 4 with 11, 6, 20-25 μm pore sizes, respectively, is hydrophilic because its hydroxyl residues on cellulose chains bond well with the oxygen residues in water. It can absorb nucleic acid particularly in the presence of high salt and alcohol concentrations, such as 1.25M NaCl and 10% polyethylene glycol (PEG), and release it in low salt concentrations, such as 10 mM Tris-HCl, pH8.0, showing similar properties to that of silica [12-13].

Other types of membranes or beads, such as Nylon (Amersham Hybond-N (neutral) transfer membrane with 0.45 μm pore size) and polyethersulfone (PES) (EMD Millipore Polyethersulfone membrane) are hydrophilic because their oxygen residues in their molecular structures facilitate hydrogen bondings with hydrogen residues in water. They share similar properties to those of silica. Therefore, they are used for isolation of nucleic acid [13-14].

### Positive Charge or Cation-Based Isolation (Anion Exchange)

The method of positively charged or cationic resin and membrane (anion exchange) is also commonly used for isolating nucleic acid. In this procedure, DNA is bound to the positive charge or cation in the presence of a low salt concentration at a low pH. DNA is eluted in the presence of a high salt concentration at a high pH [9, 15-23].

The Positively charged or cationic membrane or bead comprises a supporting media and a covalently attached positive ion or cation, such as weak anion exchanger of Diethylamine (D, or DEAE), strong anion exchanger of Quaternary ammonium (Q), and another weak anion exchanger of Diethylaminopropyl (ANX) (Table1).

TABLE 1
Functional group of anion exchangers (positively charged or ationic)

| | Example of the ionic | | Working | |
|---|---|---|---|---|
| Anion exchanger | group | Type^{a} | pH | pKa |
| Diethylamine | R—CH₂N⁺H(CH₂H₅)₂ | Weak | pH 4-10 | 9.5 |
| (D, or DEAE) | | | | |
| Quaternary | R—CH₂N⁺(CH₃)₃ | Strong | pH 2-12 | 11 |
| ammonium (Q) | | | | |
| Diethylaminopropyl | R—CH₂CHOHCH₂N⁺H | Weak | pH 4-10 | |
| (ANX) | (CH₂CH₃)₂ | | | |

Footnotes of Table 1
^{a}The terms of strong and weak refer to the extent that the ionization state of the ionic groups with pH.

The binding principle is based on the interaction between the negatively charged phosphates of the DNA backbone and the positively charged group, e.g., DEAE, on the surface. The salt concentration and pH of a solution used determine whether DNA is bound or eluted.

Positively charged or cationic bead-based columns (anion exchange), often gravity flow driven, are common, e.g., QIAGEN genomic-tips kit. The QIAGEN resin contains porous silica bead as support coated with diethylaminoethanol (DEAE) functional group. Others include Pall AcroSep chromatography columns with DEAE weak anion exchange ceramic HyperD F bead and with Q strong anion exchange ceramic HyperD F bead.

In contrast to the bead-based columns, positively charged or cationic membrane-based columns, often spin or vacuum flow driven, are available. They have the advantages of high flow rate, low cost and high throughput, e.g., Vivapure weak anion exchange mini D membrane spin column and Vivapure strong anion exchange mini Q membrane spin column [24].

Positively charged or cationic columns provide an easy, safe and reliable method for the isolation of nucleic acids from various types of samples. The prepared nucleic acid is of superior purity equivalent to two rounds of purification on CsCl gradient centrifuge.

However, the eluted nucleic acid is not ready to use because of the high salt concentration in the elution buffer. An extra step of ethanol or isopropanol precipitation is typically needed to remove the salt.

Double-Layer Chromatography and Column of Ding et al. (U.S. Pat. No. 9,163,230)

Ding et al. proved the principle of double-layer chromatography comprising a first DEAE anion exchange membrane (Waterman DE 81 paper, positively charged or cationic) and a second serially coupled silica membrane. When a DNA containing solution flows through the first DEAE membrane, the DNA binds to and then elutes from the first membrane. When the eluted solution then flows through the second silica membrane, the DNA binds to and then elutes from. As a result, the recovery rate of isolated DNA was 30% and the size ranges from >500 bp to 20 kp.

However, plasma and urine samples are characteristic of large sample volumes, small nucleic acid sizes (e.g., healthy: plasma cf-DNAbp and cf-RNA 150 nt, urine cf-DNA 80-100 bp), and low nucleic acid concentrations (e.g., healthy: plasma cf-DNA and cf-RNA 1-100 ng/mL, urine cf-DNA 1-20 ng/mL). The original column of Ding et al. didn't work efficiently for such liquid biopsy samples because their small-sized cf-DNA bound to Waterman DE 81 paper too weakly and/or were eluted too easily.

### Advantage of this Invention

The present invention discloses a deblocking double-layer chromatography and cassette for isolating nucleic acids particularly from urine sample with a large sample volume, a small nucleic acid size and a low nucleic acid concentration.

Compared with the double-layer chromatography and column of Ding et al. (U.S. Pat. No. 9,163,230) [25], through further development, we optimized the membrane compositions and the solution compositions for isolating cf-DNA and cf-RNA from liquid biopsy samples.

Furthermore, we set up the “deblocking” mechanism to overcome a problem that the membrane can be blocked by solid particles in liquid biopsy samples, thus greatly increasing its flowability. In addition, it omits a pre-filtering step in the procedure, thus particularly suitable for its automation.

### SUMMARY OF THE INVENTION

A chromatography method for isolating nucleic acids comprises: a) providing a chromatography cassette or device comprising a first solid layer of positively charged or cationic membrane or bead and a second solid layer of membrane or bead which is coupled with the first layer by a coupling solution, b) forwardly flowing a first solution (sample solution) containing a nucleic acid through the first layer to which the nucleic acid becomes bound, while solid particles in the sample solution may block the first layer to reduce its flowability, c) reversely flowing a second solution (deblocking solution) through the first layer to which the nucleic acid still remains bound, and deblocking the first layer to increase its flowability, which step b) and step c) alternate at least one time, d) forwardly flowing a third solution (coupling solution) through the first layer from which the bound nucleic acid becomes eluted, and then flowing through the second layer to which the eluted nucleic acid becomes bound, and e) forwardly flowing a fourth solution (elution solution) through the second layer from which the bound nucleic acid become eluted. In such a way, the first layer can be deblocked to increase its flowability.

The first layer of positively charged or cationic membrane or bead comprises anion exchange membrane or bead selected from the group consisting of Diethylamine (D, or DEAE), Quaternary ammonium (Q), and Diethylaminopropyl (ANX) membranes or beads.

The positively charged or cationic anion exchange membrane or bead is Quaternary ammonium (Q) membrane or bead.

The second layer of membrane or bead is selected from the group consisting of silica, silicon carbide, cellulose, neutral Nylon and polyethersulfone membranes or beads which is coupled from the first layer by the coupling solution.

The second layer of the membrane or bead is silica membrane or bead.

The sample solution comprises DNA and RNA.

The ample solution comprises cfDNA and cfRNA.

The sample solution comprises plasma and urine.

A plasma sample solution, if the viscosity is high, can be diluted before or even during the injection to reduce its viscosity.

The second solution (deblocking solution) comprises ≤200 mM salt concentration, pH 5.0-8.0.

The second solution (deblocking solution) is 1×PBS buffer.

The third solution (coupling solution) comprises chaotropic salt selected from the group consisting of guanidinium thiocyanate, guanidinium hydrochloride, sodium iodide, and sodium perchlorate.

In the third solution (coupling solution), the chaotropic salt concentration is ≥2 M.

The third solution (coupling solution) has pH 6-7.

The third solution (coupling solution) comprises ethanol or isopropanol.

The fourth solution (elution solution) comprises 10 mM Tris-HCl, 0.1 mM EDTA, pH 8.0-9.0.

The method further comprises a step between steps d and e: flowing a washing solution-2 to wash contaminants of proteins, carbohydrates and small metabolites from the second layer.

The washing solution-2 comprises ethanol or isopropanol.

The washing solution-2 comprises 2-10 mM Tris-HCl, 80%-95% ethanol, pH 6-8.

A chromatography cassette or device for isolating nucleic acids comprises: a) a first solid layer of positively charged or cationic membrane or bead and a second solid layer of membrane or bead which is coupled with the first layer by a coupling solution, b) forwardly flowing a first solution (sample solution) containing a nucleic acid through the first layer to which the nucleic acid becomes bound, while solid particles in the first solution may block the first layer and reduce its flowability, c) reversely flowing a second solution (deblocking solution) through the first layer to which the nucleic acid still remains bound, and deblocking the first layer to increase its flowability, which step b) and step c) alternate at least one time, d) forwardly flowing a third solution (coupling solution) through the first layer from which the bound nucleic acid is eluted, and then flowing the second layer to which the eluted nucleic acid becomes bound, and e) forwardly flowing a fourth solution (elution solution) through the second layer from which the bound nucleic acid is eluted. In such a way, the first layer can be deblocked to increase its flowability.

### BRIEF DESCRIPTION OF THE DRAWINGS

FIG. 1 shows a schematic of the principle of double-layer chromatography. The first solid layer is a Q positively charged or cationic membrane, and the second solid layer is a silica membrane, which are serially coupled in the order. Solutions flow through the Q membrane and then through the silica membrane, indicated by arrows. The nucleic acid, indicated by double curved lines, is bound to and then eluted from the Q membrane. Then the eluted nucleic acid is bound to and then eluted from the silica membrane.

FIG. 2 is a schematic of the improved double-layer column of this instant application for liquid biopsy samples. As the first step of the procedure, the sample solution is pre-filtered by a syringe filter, and then the filtrate is put into the column. The first layer of the column is a Q membrane and the second layer is a silica membrane in the same column. The initial input is a sample solution and the final output is an elution solution containing the nucleic acids such as cf-DNA.

FIG. 3 is a schematic of a deblocking double-layer cassette of this instant application. The first layer is a Q membrane in a first Q-cassette and the second layer is a silica membrane in a second S-cassette, which are serially coupled in the order. In the isolation process, an extra step is added that a deblocking solution is reversely flowed through the Q membrane, deblocking the micro-pores on the Q membrane and thus increasing its flowability. In addition, the sample loading step and the deblocking step alternate at least once. The initial input is a sample solution and the final output is an elution solution containing the nucleic acids such as cf-DNA.

### DETAILED DESCRIPTION OF THE INVENTION

### Terminology of Chromatography

Unless defined otherwise, all technical and scientific terms used herein have the same meaning as commonly understood by one of ordinary skill in the art.

Chromatography is a process or method for separating components of a mixture.

Nucleic acids comprise DNA and RNA, which may be naked or embraced by proteins.

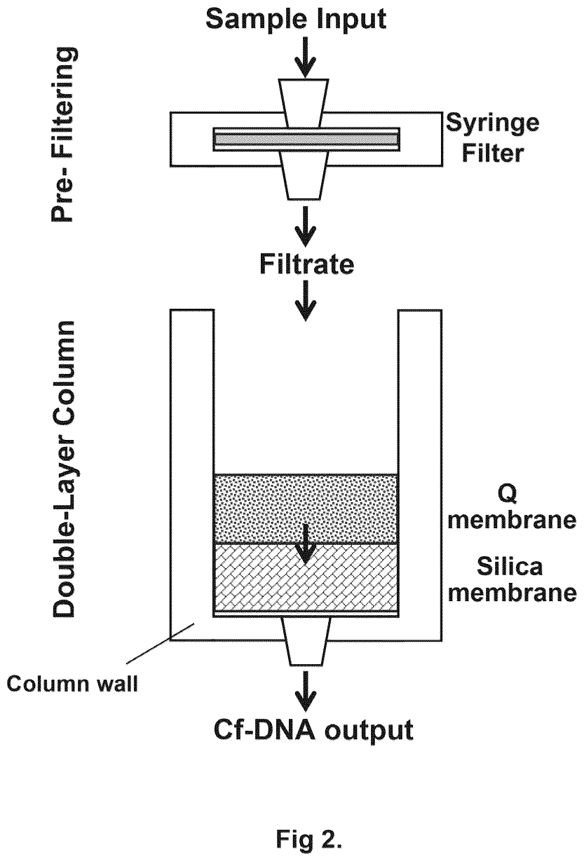

Column means an upright pillar, typically cylindrical, such as schematic diagram in FIG. 2.

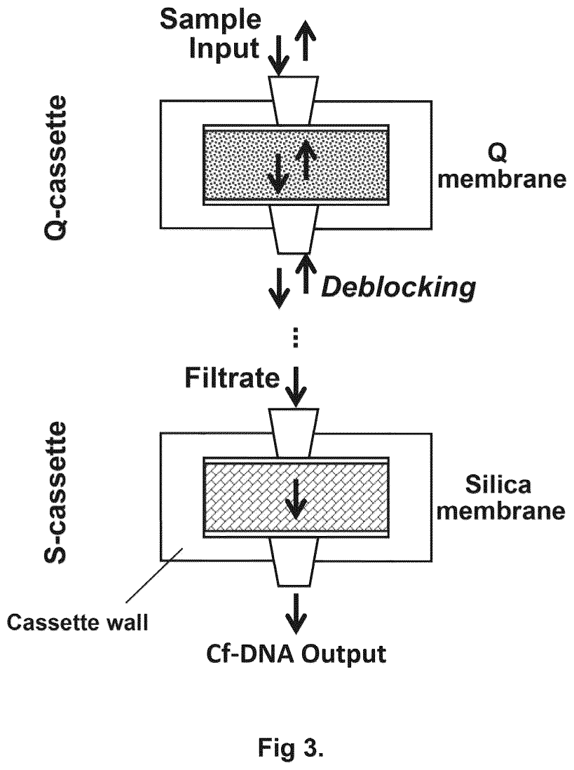

Cassette means a little box with chamber(s), such as schematic diagrams in FIG. 3.

Flowability of a membrane means its ability for fluid and loose particulate powder to flow through.

Cf-DNA means cell free DNA in plasma and urine.

Cf-RNA means cell free RNA in plasma and urine.

Liquid biopsy samples comprise plasma and urine which are characteristic of large sample volumes, low nucleic acid concentrations, and small nucleic acid sizes as small as 80-100 bp.

Solid particles in plasma are mainly micro-clots of blood cell with various amounts and sizes.

Solid particles in urine are mainly crystals as well as dead cells with various amounts and sizes. The compositions of such crystals include ammonium biurate, bilirubin, calcium oxalate or calcium phosphate, cystine, hippuric acid, leucine, struvite (magnesium ammonium phosphate), tyrosine, uric acid, and xanthine.

### Terminology of PCR and PAP

PCR refers to polymerase chain reaction.

RT-PCR refers to reverse transcription polymerase chain reaction, which includes two stages of reverse transcription and then polymerase chain reaction.

Polymerase refers to a polymerase characterized as polymerization or extension of deoxyribonucleic acid.

Reverse transcriptase refers to a polymerase characterized as catalyzing DNA polymerization or extension on RNA template.

PAP refers to pyrophosphorolysis activated polymerization.

RT-PAP refers to reverse transcription pyrophosphorolysis activated polymerization, which includes two stages of reverse transcription and then pyrophosphorolysis activated polymerization.

PAP polymerase is a genetic thermostable engineered form of polymerase containing F667Y amino acid changes. It has 5′-3′ polymerase activity and pyrophosphorolysis activity.

Pyrophosphorolysis is the reverse reaction of deoxyribonucleic acid polymerization. In the presence of pyrophosphate, the 3′ nucleotide is removed by a polymerase from duplex DNA to generate a triphosphate nucleotide and a 3′ unblocked duplex DNA: [dNMP]ₙ+PPi→ [dNMP]ₙ₋₁+dNTP (Deutscher and Kornberg, 1969).

3′ blocked primer refers to an oligonucleotide with a 3′ non-extendable nucleotide (3′ blocker), such as a dideoxynucleotide or an acycolonucleotide. The 3′ nucleotide could not be directly extended, but it can be removed by pyrophosphorolysis and then the unblocked primer can be extended by polymerase.

In realtime PCR and PAP detection, baseline is the level of fluorescence signal during initial cycles. The low level can be considered as background or “noise” of the reaction.

Threshold is defined as the level of fluorescence signal that is a significant higher than baseline signal and can distinguish amplification signal from the background.

Ct (threshold cycle) is the cycle number at which the fluorescence signal crosses the threshold. It is used to calculate starting template amount or concentration of PCR or PAP.

### Example 1. Materials and Methods

### Preparation of Membrane, Column and Cassette

A column with 7 mm inner diameter was made of polypropylene, provided by A-Gen Biotechnology Limited, China.

A cassette, a reusable syringe filter device with 25 mm or 13 mm inner diameter, was made of polypropylene, provided by Ks-Tek, China.

Q anion exchange PVDF membrane (Q membrane, positively charged)) was quaternary ammonium positive charged with 1 μm pore diameter and 120 μm thickness, provided by Cobetter Filtration Equipment, China.

Whatman Grade GF/B glass microfiber filters (Silica membrane) was with lum pore diameter and 675 μm thickness, provided by Cytiva Corporation, USA.

### Preparation of Primers for PAP and RT-PCR

In order to quantify DNA and RNA amounts, PAP and RT-PCR assays were designed.

Each 3′ blocked primer, blocked with ddCMP at the 3′ end, was chemically synthesized and HPLC purified by Integrated DNA Technologies. The regular DNA primers were also synthesized by the same vendor (Table 2).

TABLE 2
List of primers and assays

| | | Sequence (5′ to 3′) | | Product | Starting |
|---|---|---|---|---|---|
| Assay | Name^{e} | (SEQ ID NO:) | Location | size (bp) | template |
| I. PAP^{a} | 28S rDNA- | TGGGTATAGGGGCGAAA | ddCMP | 66 | Genomic |
| | (9537)D | GACTAATCGAACddC^{d} (1) | | | DNA |
| | 28S rDNA- | CTGAGGGAAACTTCGGA | ddCMP | | |
| | (9602)U | GGGAACCAGCTAddC (2) | | | |
| II. | ACTB(426) | CAACCGCGAGAAGATGA | Exons 3 | 63 | Total |
| RT-PCR^{b} | 25D | CCCAGATC (3) | and 4 | | RNA |
| | ACTB(488) | GCAACGTACATGGCTGGG | Exon 4 | | |
| | 25U | GTGTTGA (4) | | | |

Footnoots of Table 2.
^{a}This PAP is chosen because it has high sensitivity to detect a little amount of the targeted human genomic DNA template.
^{b}In order to avoid non-specific amplification from potentially contaminated genomic DNA, RT-PCR primers were designed to anneal to regions that span introns of the human ACTB gene.
^{c}For example, ACTB means the human ACTB gene; (426), 5′ end of the primer begins at nucleotide 426; D, downstream (i.e., in the direction of transcription). The ACTB mRNA is from GenBank accession: NM_001101.3.
^{d}Due to the availability of chemical synthesis, ddCMP was exampled.

### Preparation of Simulated Cf-DNA Sample

Genomic DNA was extracted from blood white cells using QIAamp Blood Mini Kit according to Qiagen's protocol. The concentration of the genomic DNA was determined by UV absorbance at 260 nm.

For simulation of cf-DNA in liquid biopsy samples, the extracted genomic DNA was treated with supersonic to reduce segment sizes to 150 bp and diluted into 1×PBS buffer.

### Preparation of Simulated Cf-RNA Sample

Total RNA was extracted from human blood white cells using QIAamp RNA kit according to Qiagen′ protocol (QIAamp RNA Blood Mini Handbook). Within the process, RNase-Free DNase was used to remove contaminated genomic DNA. The concentration of the total RNA was measured by a spectrophotometer at 260 nm.

For simulation of cf-RNA in liquid biopsy samples, the extracted RNA was treated with supersonic to reduce segment sizes to 100-200 nt and diluted into 1×PBS buffer.

### Collection of Urine Sample

Each urine sample was collected into a 50 mL tube with EDTA at a final concentration of 10 mM and stored at −20° C. until use.

### PAP Reaction to Measure Cf-DNA Amount

A reaction mixture of 20 μl contained 50 mM Tris-HCl (pH 8.0 at 25° C.), 6 mM (NH₄)₂SO₄, 1.2 mM MgCl₂, 25 μM each dNTPs (dATP, dTTP, dGTP and dCTP), 0.1 μM each primers, 90 μM Na₄PPᵢ, 1 units of PAP polymerase, 0.2× SybrGreen I dye and 1 or 5 μl of DNA.

A Bio-Rad CFX96 real-time PCR detection system was used for quantification of the amplified product. Analysis mode: fluorophore, Baseline setting: baseline subtracted curve fit, Threshold cycle (Ct) determination: single threshold, Baseline method: Auto calculated, Threshold setting: auto calculated. Ct value was thus measured for each reaction which is proportional to the amount of amplified product in the early exponential phase of amplification.

A PAP cycling program entailed 95° C. for 10 seconds, 60° C. for 30 seconds, 64° C. for 30 seconds, and 68° C. for 30 seconds for a total of 40 cycles. A denaturing step of 96° C. for 2 min was added before the first cycle. To confirm the amplified product, melting curve analysis was followed from 60° C. to 95° C. with increment 1° C. and holding 5 seconds.

### Set Up a Positive Standard Curve of Cf-DNA for Quantification

PAP was used to quantify cf-DNA amount. A positive standard curve of cf-DNA was made by 10-fold serial diluted to 5 ng, 1 ng, 0.5 ng, 0.05 ng of cf-DNA in a 20 μl reaction with each condition repeated three times.

After amplification, the Ct was obtained under each condition and averaged. Plotted the 1 g (DNA amount) vs. the average Ct, and got the linear regression equation and the correlation coefficient R².

### One-Step RT-PCR to Measure Cf-RNA Amount

An one-step RT-PCR reaction mixture of 20 μl contained 50 mM Tris-HCl (pH 8.3 at 25° C.), 6 mM (NH₄)₂SO₄, 1.5 mM MgCl₂, 200 μM each dNTPs (dATP, dTTP, dGTP and dCTP), 0.1 μM each primer of RT-PCR Assay I, 0.2× SybrGreen I dye, 0.02% Twee-20, 0.5 U of Thermophilic Reverse Transcriptase, 1 U of Taq-Fast DNA polymerase and 1 ul of RNA.

A Bio-Rad CFX96 real time PCR detection system was used for quantification of RNA amount. The one-step RT-PCR cycling program entailed 60° C. for 10 minutes for reverse transcription; then 95° C. for 2 minutes for heat-inactivation of reverse transcriptase activity; and then cycling 95° C. for 10 seconds, 60° C. for 30 seconds and 72° C. for 60 seconds for 35 cycles. To confirm the amplified product, melting curve analysis was followed from 60° C. to 95° C. with increment 1° C. and holding 5 seconds.

### Set Up a Positive Standard Curve of Cf-RNA for Quantification

RT-PCR was used to quantify cf-RNA amount. A positive standard curve of cf-RNA was made by 10-fold serial diluted to 5 ng, 1 ng, 0.5 ng, 0.05 ng of cf-RNA in a 20 μl reaction with each condition repeated three times.

After amplification, the Ct was obtained under each condition and averaged. Plotted the Ig (RNA amount) vs. the average Ct, and got the linear regression equation and the correlation coefficient R².

### Example 2. Improved Double-Layer Chromatography Column

### Development of the Improved Column

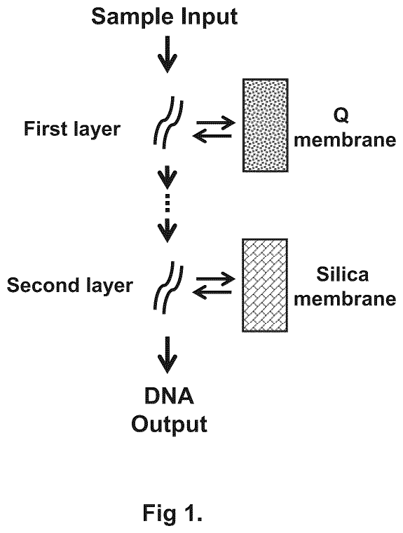

Based on the principle of double-layer chromatography (FIG. 1) of Ding et al., through further development, we optimized its membrane compositions and solution compositions of the improved column, greatly improving the performances for isolating cf-DNA and cf-RNA from liquid biopsy samples.

Specifically, the improved performances include:
1) Plasma volume: 5 ml, urine volume: 50 ml.
2) DNA purity: directly used for downstream applications, such as NGS (Next Generation Sequencing), PCR (Polymerase Chain Reaction) and PAP (Pyrophosphorolysis Activated Polymerization).
3) DNA size: mainly in range of 80 bp to 500 bp. Large DNA fragments of 500 bp-20 kp from lysed white cells remain on the membrane without eluting.
4) DNA recovery rate: >90%.
5) DNA capability: >12 μg.

The different features between the improved column and the original column of Ding et al. are compared (Table 3).

TABLE 3
Comparison of Ding et al. original column and the improved column

| Feature | Ding et al. original column | The improved column |
|---|---|---|
| Targeted nucleic | Genomic DNA of blood | cf-DNA/cf-RNA of |
| acid | and tissue | plasma and urine |
| First layer of | DEAE membrane (D) | Quaternary ammonium |
| double-layer column | | membrane (Q) |
| Recovery rate | 30% | >90% |
| Recovered DNA size | Large fragment >500 bp | As small as 80-100 bp |

As a result, we developed two products for plasma and urine samples (Human plasma cell-free DNA extraction kit and Human urine cell-free DNA extraction kit).

### The Compositions of the Improved Column

### Positively Charged or Cationic Membrane or Bead of the First Solid Layer

We tested many types of positively charged or cationic membranes and beads under various solution conditions, including Waterman DEAE anion exchange cellulose membrane (DE81 paper), Vivapure DEAE anion exchange regenerated cellulose membrane, Pall DEAE Ceramic HyperD F bead, Qiagen DEAE anion exchange bead, Pall Mustang Q anion exchange cross-linked polymeric membrane, Pall Biodyne Nylon B membrane (Strongly positively charged Q membrane with strong anion exchanger Q, Nylon 6,6 media, pore size 0.8 μm), Pall Biodyne Nylon Plus membrane (Strongly positively charged membrane with strong anion exchanger which has an extremely high isoelectric point, Nylon 6,6 media, pore size 0.45 μm), and Cobetter Q anion exchange PVDF membrane (Strongly positively charged Q membrane with strong anion exchanger Q, PVDF media, pore size 1.0 μm).

The criteria to choose positively charged or cationic membrane or bead of the first layer included:
1) Binding efficiency: cf-DNA and cf-RNA bind to the first layer efficiently.
2) Elution efficiency: The bound nucleic acids are eluted by a coupling solution efficiently (Table 4), which is affected by its compositions of Guanidine, buffer, alcohol, and pH of the solution.
3) Coupling efficiency from the first layer to the second layer: Through a coupling solution, the bound nucleic acids are not only efficiently eluted from the first layer but also efficiently bound to the second layer.
4) Flowability: The solutions flow through the first layer efficiently, which is affected by the micropore size, thickness, and area of the first layer.
5) Deblocking efficiency: A deblocking solution reversely flows through and deblocks the first layer to increase its flowability efficiently, once the first layer is blocked by solid particles in plasma and urine, which is affected by the micropore size, thickness, and area of the first layer. At the same time, the deblocking solution does not elute the bound nucleic acids on the first layer.

As testing results, we chose Cobetter Q anion exchange PVDF membrane (Strongly positively charged Q membrane with strong anion exchanger Q, PVDF media, pore size 1.0 μm) for liquid biopsy samples.

### Membrane or Bead of the Second Solid Layer

We tested many silica membranes and beads under various solution conditions, including EZgene plasmid purification miniprep kit from Bioland, Pure Yield plasmid midiprep system from Promega, Wizard plus minipreps DNA purification system from Promega, QIAprep miniprep kit from Qiagen, Whatman Grade GF/F glass microfiber filters, and Whatman Grade GF/B glass microfiber filters (Silica membrane).

Besides silica, other types of membranes or beads of silicon carbide, cellulose, neutral Nylon and polyethersulfone, can absorb nucleic acid in a condition such as high salt and alcohol concentrations and release it in a different condition such as low salt concentrations of 10 mM Tris-HCl, pH 8.0, 0.1 mM EDTA, showing similar properties to that of silica. Therefore, they can function in similar ways of silica to be coupled with the first layer by a coupling solution.

The criteria to choose the membrane or bead of the second layer included:
1) Binding efficiency: cf-DNA and cf-RNA bind to the second layer efficiently using the same coupling solution, which is affected by the same compositions of Guanidine, buffer, alcohol, and pH of the coupling solution (Table 4).
2) Elution efficiency: The bound nucleic acids are eluted efficiently by an elution solution (Table 4), which is affected by its compositions of salt strength, buffer concentration, and pH of the elution solution.
3) Coupling efficiency from the first layer to the second layer: Through the same coupling solution, the bound nucleic acids are not only efficiently eluted from the first layer but also efficiently bound to the second solid layer.
4) Flowability: The solutions pass through the second layer efficiently, which is affected by the micropore size, thickness, and area of the second layer.

As testing results, we chose Whatman Grade GF/B glass microfiber filters (Silica membrane).

### Solutions

Solutions, particularly the coupling solution, were gradually evolved from Ding et al. (U.S. Pat. No. 9,163,230) to their current versions in Table 4 for liquid biopsy samples. Compositions of Guanidine (GuCI), buffer (Bis-Tris), alcohol (ethanol and isopropanol), and pH were extensively explored and tested under various membrane conditions.

The criteria to choose a solution included:
Cf-DNA and cf-RNA in the sample solution(S) bind to the Q membrane efficiently.

The washing solution-1 (W-1) washes the Q membrane but not elutes the nucleic acids.

The coupling solution (C) elutes the nucleic acids from the Q membrane efficiently and then binds the eluted nucleic acids to the silica membrane efficiently.

Washing solution-2 (W-2) washes the silica membrane but not elutes the nucleic acids.

The elution solution (E) elutes the nucleic acids from the silica membrane efficiently.

In addition, if a deblocking cassette is used, the deblocking solution (D) reversely flows through and deblocks the Q membrane to increase its flowability, which is discussed in the next section.

Furthermore, the compositions were adjusted for isolating cf-DNA as well as cf-RNA.

TABLE 4
Compositions and functions of the aqueous solutions

| | | Function of | Function of |
|---|---|---|---|
| | | the improved | the deblocking |
| Solution | Composition | column | cassette |
| Deblocking | 1xPBS buffer (137 mM | Not used | Deblocking the |
| solution (D) | NaCl, 2.7 mM KCl, | | Q membranes |
| | 4.3 mM, Na₂HPO, | | to increase its |
| | 1.47 mM KH₂PO, pH | | flowability |
| | 7.4) | | |
| Washing | 150 mM NaCl, 100 mM | Washing the Q | Not used |
| solution-1 | BisTris-HCl, pH 6.5, | membrane | |
| (W-1) | 40% isopropanol | | |
| Coupling | 4M GuHCl, 100 mM | Elute nucleic | The same |
| solution (C) | BisTris-HCl, pH 6.5, | acid from the Q | |
| | 40% isopropanol | membrane and | |
| | | bind the eluted | |
| | | nucleic acid to | |
| | | the silica | |
| | | membrane | |
| Washing | 2 mM Tris-HCl, pH | Washing the | The same |
| solution-2 | 8.0, 95% ethanol | silica membrane | |
| (W-2) | | | |
| Elution | 10 mM Tris-HCl, | Elute the nucleic | The same |
| solution (E) | 0.1 mM EDTA, | acid from the | |
| | pH 8.0 | silica membrane | |

### Isolation of Cf-DNA and Cf-RNA Using the Improved Column

### Preparation of the Improved Column

The Q membrane was made of PVDF and positive charged by quaternary ammonium with 1 μm pore diameter and 120 μm thickness. The silica membrane was made of glass microfiber with 1 μm pore diameter and 675 μm thickness.

Took a column made of polypropylene with 7 mm inner diameter. Cut the Q membrane and the silica membrane each with 7 mm diameter.

Assembled and sealed two pieces of the Q membrane (upper) and two pieces of the silica membrane (lower) into the column (FIG. 2).

### Procedure for Cf-DNA and Cf-RNA Isolation

The following procedure was used:
1. Pre-filter 5 mL of plasma, 50 mL of urine or an alternative sample with a syringe filter (PES, 0.22 μm pore size and 25 mm diameter), and collect the filtrated sample (Stop filtering when the sample is no longer manually pushed through the filter and replace with another new syringe filter).
2. Transfer the filtrated sample into the column. Start vacuum and flow the filtrated sample through the column at a velocity of 1-5 mL/min. Discard the flowed-out filtrate.
4. Add 0.7 mL of the washing solution-1 (W-1) into the column. Vacuum and flow the W-1 solution through the column. Discard the flowed-out filtrate.
5. Add 0.7 mL of the coupling solution (C) into the column. Vacuum and flow the C solution through the column. Discard the flowed-out filtrate.
6. Add 1.4 mL of the washing solution-2 (W-2) into the column. Vacuum and flow the W-2 solution through the column. Discard the flowed-out filtrate. To remove minimal ethanol left within the column, centrifuge the column at 8,000 g for 1 minute and discard the flowed-out filtrate.
7. Add 200 μL of the elution solution (E) into the column and wait for 3 minutes at room temperature. Centrifuge at 8,000 g for 1 minute to collect the flowed-out E solution. Results of the improved column are discussed in a later section.

### Example 3. Deblocking Double-Layer Chromatograpg and Cassette

A Problem of the Improved Column with Plasma and Urine Samples

With the improved double-layer column, a “blocking” problem sometimes occurs with plasma and urine samples. For example, if urine or plasma samples are directly added to the double-layer column, the micropores on the membranes are easily blocked by the large amounts of solid particles.

It cautions in our instruction: “Urine and plasma samples must be pre-filtered to remove large amounts of solid particles”. As the first step of the isolation, pre-filtering must be manipulated for the urine as well as plasma samples with a syringe pre-filter to remove the solid particles. Even so, the syringe pre-filtering step and even the following double-layer column step are still blocked sometimes if unexpected large amounts of solid particles.

Another problem of the improve column is automation. Due to the pre-filtering step, it is more difficult to integrate this step into an automated process.

### Development of the Deblocking Cassette

In order to overcome the “blocking” problem which occurs with liquid biopsy samples, we introduced the deblocking mechanism into double-layer chromatography and cassette (FIG. 3).

The cassette comprises the first Q membrane (Q cassette) and the second silica membrane (S cassette). In the isolation process, an extra step of deblocking is introduced that a deblocking solution is reversely flowed through the Q membrane, deblocking the micro-pores on the Q membrane and thus increasing its flowability. In addition, the sample loading step and the deblocking step alternate at least once.

### Isolation of Cf-DNA and Cf-RNA Using the Deblocking Cassette

### Preparation of the Deblocking Cassette

Took two cassette devices (reusable syringe filters) made of polypropylene with 25 mm and 13 mm inner diameters, respectively. Cut the Q membrane with 25 mm diameter and the silica membrane with 13 mm diameter.

Q-cassette: Assembled and sealed two pieces of the Q membrane into the first cassette with 25 mm diameter.

S-cassette: Assembled and sealed two pieces of the silica membrane into the second cassette with 13 mm diameter (FIG. 3).

### Procedure for Cf-DNA and Cf-RNA Isolation

1. Use a 1^{st} syringe to take 5 mL of plasma, 50 mL of urine or an alternative sample.

2. Connect to the up entrance of the Q-cassette, inject 2 mL of plasma or 10 mL of urine samples into the Q-cassette at a velocity of 1-5 mL/min. Collect the 1^{st} filtrate from the low exit.

3. Use a 2^{nd} syringe to take the deblocking solution. Connect to the low exit of the Q-cassette. Reversely inject 20 mL of the D solution into the Q-cassette. Collect the 2^{nd} filtrate from the up entrance.

4. Alternate step 2 and step 3 at least one time until all the sample solution is injected.

5. Use a 3^{rd} syringe to take the coupling solution. Connect to the up entrance of the Q-cassette. Inject 2 mL of the C solution into the Q-cassette. Collect the 3^{rd} flowed-out C solution from the low exit by a 4^{th} syringe.

6. Connect the 4^{th} syringe to the up entrance of the S-cassette. Inject the collected 2 mL of the C solution (the 3rd flowed-out) into the S-cassette. Collect the 4th filtrate from the low exit.

7. Use a 5^{th} syringe to take the washing solution-2. Connect to the up entrance of the S-cassette. Inject 2 mL of the W-2 solution into the S-cassette. Collect the 5th filtrate from the low exit. To remove minimal ethanol left within the S-cassette, centrifuge at 3,000 g for 1 minute and discard the filtrate from the low exit.

8. Use a 6^{th} syringe to take the elution solution (E). Connect to the up entrance of the S-cassette. Inject 0.2 mL of the E solution into the S-cassette and wait for 3 minutes at room temperature. Centrifuge at 3,000 g for 1 minute to collect the 6th flowed-out E solution form the low exit.

Extraction of Cf-DNA and Cf-RNA Samples by the Deblocking Cassette and in Comparison with the Improved Column

The simulated cf-DNA sample was prepared, taking similar characteristics of cf-DNA in plasma and urine, i.e., the large volume 20 mL, the small size ˜150 pb, and the low concentration 10 ng/mL.

The cf-DNA was isolated by the deblocking cassette in comparison with the improved column according to the above procedures. As a result, the cf-DNA was eluted into 200 μL of the elution solution (TE buffer).

To quantify the eluted cf-DNA amount, PAP was used to amplify a 65 bp fragment of the human rDNA gene (the rDNA gene has 1000 copies per genome and thus it is quantified more easily). Ct value was obtained, averaged and correlated to that of the cf-DNA standard control. Then the recovery rate was thus calculated (Table 5).

The simulated cf-RNA sample was also prepared (20 mL, 100-200 nt, 10 ng/ml). The cf-RNA was isolated by the deblocking cassette which was compared with the improved column according to the above procedures. Finally, the cf-RNA was eluted into 200 μL of TE buffer.

To quantify the eluted cf-RNA amount, RT-PCR was used to amplify a 63 nt mRNA transcript of the human ACTB gene (the mRNA is expressed abundantly and thus it is quantified more easily). Ct value was obtained, averaged and correlated to that of the cf-RNA standard control. Then the recovery rate was thus calculated (Table 5).

Compared with the improved column, the deblocking cassette worked effectively to isolate the cf-DNA and cf-RNA, thus the deblocking step did not affect the recovery rates substantially.

TABLE 5
Comparison between the improved column and the deblocking cassette

| | | The improved | |
|---|---|---|---|
| Feature | | column | The deblocking cassette |
| Sample volume | | 20 mL | 20 mL |
| Pre-filtering | | Syringe filter | — |
| | | (0.22 um pore size | |
| | | and 25 mm diameter) | |
| Injecting sample | | One time into the | Alternate four times into |
| | | column | the Q-cassette (5 ml, 5 mL, |
| | | | 5 mL and 5 mL) |
| Deblocking | | — | Alternate four times (20 ml, |
| (reversely injecting) | | | 20 mL, 20 mL and 20 mL) |
| cf-DNA | Ct of 1 ul | Average 19.3 | Average 19.3 |
| by | elution | | |
| PAP^{a} | Recovery | 93.1% | 91.0% |
| | rate | | |
| cf-RNA | Ct of 1 ul | Average 16.2 | Average 16.3 |
| by | elution | | |
| RT-PCR^{b} | Recovery | 85.5% | 81.2% |
| | rate | | |

Footnotes of Table 5.
^{a}cf-DNA quantification by PAP: A positive standard curve of cf-DNA was made by 10-fold serial diluted to 5 ng, 1 ng, 0.5 ng, 0.05 ng of cf-DNA in a 20 ul reaction with each condition repeated three times. For cf-DNA samples, each Ct value was averaged from three identical amplifications to 16.8, 20.2 and 23.7, respectively, and used to deduce a linear regression equation of lg(DNA) = −0.29 × Ct + 5.56 with the correlation coefficient R² of 0.99.
^{b}cf-RNA quantification by RT-PCR: For positive standard control, 5 ng, 1 ng, 0.5 ng, 0.05 ng of cf-RNA in a 20 ul reaction were prepared with each condition repeated three times. For cf-DNA samples, each Ct value was averaged from three identical amplifications to 13.5, 17.0 and 20.6 and used to derive a linear regression equation of lg(RNA) = −0.28 × Ct + 4.50 with the correlation coefficient R² of 0.99.

Extraction of Urine Sample by the Deblocking Cassette in Comparison with the Improved Column

Ultimately, 50 mL of urine was prepared. The cf-DNA was isolated by the deblocking cassette which was compared with the improved column according to the above procedures. As a result, the cf-DNA was eluted into 200 μL of TE buffer.

For the deblocking cassette, the effect of deblocking was observed to increase the flowability of the Q membrane. If the urine sample was directly injected into the Q cassette without the deblocking step, the Q membrane was blocked with 25-30 mL of the urine sample. However, when the alternating deblocking step was applied, the Q membrane was not blocked with 50 mL of the urine sample (Table 6).

For quantification of the cf-DNA amount, PAP was used to amplify a 65 bp fragment of the human rDNA gene (1000 copies per genome). The Ct value was obtained, averaged and related to that of the cf-DNA standard control. Then the total amount cf-DNA recovered from 50 mL of the urine sample was calculated (Table 6).

Compared with the improved column, the deblocking cassette worked effectively with 50 mL of the urine sample.

TABLE 6
Comparison between the improved column and the deblocking cassette
for isolating urine samples

| Feature | The improved column | The deblocking cassette |
|---|---|---|
| Urine volume | 50 mL | 50 mL |
| Pre-filtering | Syringe filter | — |
| | (0.22 um pore size | |
| | and 25 mm diameter) | |
| Difficulty of | High. Push hard. | — |
| pre-filtering | Sometimes need two | |
| | syringe filters to finish | |
| Injecting sample | One time into the | Alternate five times into the |
| (forwardly) | column | Q-cassette (10 ml, 10 mL, |
| | | 10 mL, 10 mL and 10 mL) |
| Difficulty of | High, blocked | Medium |
| injecting | | |
| Deblocking | — | Alternate five times |
| (reversely injecting) | | (20 ml, 20 mL, 20 mL, |
| | | 20 mL and 20 mL) |
| Difficulty of | — | Low |
| deblocking | | |
| Ct of cf-DNA | Average 17.2 | Average 17.4 |
| from 5 ul elution | | |
| Total amount | 148 ng | 130 ng |
| recovered from | | |
| 50 ml of urine | | |

### REFERENCE

1. Vogelstein, B., and Gillespie, D. (1979). Preparative and analytical purification of DNA from agarose. Proceedings of the National Academy of Sciences of the United States of America 76, 615-619.
2. Chen, C. W., and Thomas, C. A., Jr. (1980). Recovery of DNA segments from agarose gels. Analytical biochemistry 101, 339-341.
3. Marko, M. A., Chipperfield, R., and Birnboim, H. C. (1982). A procedure for the large-scale isolation of highly purified plasmid DNA using alkaline extraction and binding to glass powder. Analytical biochemistry 121, 382-387.
4. Boom, R., Sol, C. J., Salimans, M. M., Jansen, C. L., Wertheim-van Dillen, P. M., and van der Noordaa, J. (1990). Rapid and simple method for purification of nucleic acids. Journal of clinical microbiology 28, 495-503.
5. Boom, R., Sol, C., Beld, M., Weel, J., Goudsmit, J., and Wertheim-van Dillen, P. (1999). Improved silica-guanidiniumthiocyanate DNA isolation procedure based on selective binding of bovine alpha-casein to silica particles. Journal of clinical microbiology 37, 615-619.
6. Tian, H., Huhmer, A. F., and Landers, J. P. (2000). Evaluation of silica resins for direct and efficient extraction of DNA from complex biological matrices in a miniaturized format. Analytical biochemistry 283, 175-191.
7. Breadmore, M. C., Wolfe, K. A., Arcibal, I. G., Leung, W. K., Dickson, D., Giordano, B. C., Power, M. E., Ferrance, J. P., Feldman, S. H., Norris, P. M., et al. (2003). Microchip-based purification of DNA from biological samples. Analytical chemistry 75, 1880-1886.
8. Padhye, V. V., York, C., and Burkiewicz, A. (1997). Nucleic acid purification on silica gel and glass mixtures. U.S. Pat. No. 5,658,548.
9. Smith, C. E., Holmes, D. L., Simpson, D. J., Katzenhendler, J., Bitner, R. M., and Grosch, J. C. (2002). Mixed-bed solid phase and its use in the isolation of nucleic acids. U.S. Pat. No. 6,376,194.
10. Thompson, J. D., Cuddy, K. K., Haines, D. S., and Gillepsie, D. (1990). Extraction of cellular DNA from crude cell lysate with glass. Nucleic acids research 18, 1074.
11. Melzak, K. A., Sherwood, C. S., Turner, R. F. B., and Haynes, C. A. (1996). Driving Forces for DNA Adsorption to Silica in Perchlorate Solutions. J Colloid and Interface Science 181, 635-644.
12. Nargessi, R. D., and Pourfarzaneh M. (2007). Isolation and purification of nucleic acids. U.S. Pat. No. 7,264,927.
13. Shi R, Panthee D (2017) A novel plant DNA extraction method using filter paper-based 96-well spin plate. Planta 246:579-584.
14. Mandal, G., Das, S., and Sriram Padmanabhan, S. (2018) Development of a Membrane-Based Method for Isolation of Genomic DNA from Human Blood Journal of Biomolecular Techniques 29:46-53
15. Ion Exchange Chromatography and Chromatofocusing: Principles and Methods, GE Healthcare. http://wwwgelifesciencescom/gehcls_images/GELS/Related % 20Content/Files/1314823637792/litdoc11000421AB_20110901010317pdf.
16. Teeters, M. A., Conrardy, S. E., Thomas, B. L., Root, T. W., and Lightfoot, E. N. (2003). Adsorptive membrane chromatography for purification of plasmid DNA. Journal of chromatography A 989, 165-173.
17. Endres, H. N., Johnson, J. A., Ross, C. A., Welp, J. K., and Etzel, M. R. (2003). Evaluation of an ion-exchange membrane for the purification of plasmid DNA. Biotechnology and applied biochemistry 37, 259-266.
18. Zhang, S., Krivosheyeva, A., and Nochumson, S. (2003). Large-scale capture and partial purification of plasmid DNA using anion-exchange membrane capsules. Biotechnology and applied biochemistry 37, 245-249.
19. Stadler, J., Lemmens, R., and Nyhammar, T. (2004). Plasmid DNA purification. The journal of gene medicine 6 Suppl 1, S54-66.
20. Yang, Y., Hebron, H. R., and Hang, J. (2008). High performance DNA purification using a novel ion exchange matrix. Journal of biomolecular techniques: JBT 19, 205-210.
21. Smith, C. E., Holmes, D. L., Simpson, D. J., Katzenhendler, J., Bitner, R. M., and Grosch, J. C. (2001). pH dependent ion exchange matrix and method of use in the isolation of nucleic acids. U.S. Pat. No. 6,310,199
22. Skagestad, V. (2011). Nucleic acid extraction method. U.S. Pat. No. 8,067,579.
23. Kutner, R. H., Puthli, S., Marino, M. P., and Reiser, J. (2009). Simplified production and concentration of HIV-1-based lentiviral vectors using HYPERFlask vessels and anion exchange membrane chromatography. BMC biotechnology 9, 10.
24. Ashby, M. K., Mullineaux, C. W. Rapid Purification of High Molecular Weight Bacterial Chromosomal DNA using the Vivapure 500 and Vivapure 20-D anion exchange membrane devices. Vivascience application note http://sartoriusorkr/Vivascience/Application_Notes/pdf/ASH_IE_0124pdf.
25. Ding, SF., Liu, Q. (2015). Chromatography column and method for isolating nucleic acid. U.S. Pat. No. 9,163,230.

1. A chromatography method for isolating nucleic acids, comprising:
a) providing a chromatography cassette or device comprising a first solid layer of positively charged or cationic membrane or bead, and a second solid layer of membrane or bead which is coupled from the first layer by a coupling solution,
b) forwardly flowing a first solution (sample solution) containing a nucleic acid through the first layer to which the nucleic acid becomes bound, while solid particles in the sample solution may block the first layer to reduce its flowability,
c) reversely flowing a second solution (deblocking solution) through the first layer to which the nucleic acid still remains bound, and deblocking the first layer to increase its flowability, wherein step b) and step c) alternate at least once,
d) forwardly flowing a third solution (coupling solution) through the first layer from which the bound nucleic acid becomes eluted, and then flowing through the second layer to which the eluted nucleic acid becomes bound, and
e) forwardly flowing a fourth solution (the elution solution) through the second layer from which the bound nucleic acid become eluted,
whereby, the first layer can be deblocked to increase its flowability.

2. The method of claim 1, wherein the first layer of the positively charged or cationic membrane or bead is selected from the group consisting of Diethylamine (D, or DEAE), Quaternary ammonium (Q), and Diethylaminopropyl (ANX) membranes or beads.

3. The method of claim 1, wherein the positively charged or cationic membrane or bead is Quaternary ammonium (Q) membrane or bead.

4. The method of claim 1, wherein the second layer of the membrane or bead is selected from the group consisting of silica, silicon carbide, cellulose, Nylon and polyethersulfone membrane or bead, which is coupled from the first layer by the coupling solution.

5. The method of claim 1, wherein the second layer of the membrane or bead is silica membrane or bead.

6. The method of claim 1, wherein the sample solution comprises DNA and RNA.

7. The method of claim 1, wherein the sample solution comprises cfDNA and cfRNA.

8. The method of claim 1, wherein the sample solution comprises plasma and urine.

9. The method of claim 1, wherein the second solution (deblocking solution) has ≤200 mM salt concentration.

10. The method of claim 1, wherein the second solution (deblocking solution) has pH 5.0-8.0.

11. The method of claim 1, wherein the second solution (deblocking solution) is 1×BPS buffer.

12. The method of claim 1, wherein the third solution (coupling solution) comprises chaotropic salt selected from the group consisting of guanidinium thiocyanate, guanidinium hydrochloride, sodium iodide, and sodium perchlorate.

13. The method of claim 1, wherein the third solution (coupling solution) comprises chaotropic salt has a concentration ≥2 M.

14. The method of claim 1, wherein the third solution (coupling solution) has pH 6-7.

15. The method of claim 1, wherein the third solution (coupling solution) comprises ethanol or isopropanol.

16. The method of claim 1, wherein the fourth solution (elution solution) has pH 7.0-9.0.

17. The method of claim 1, wherein the fourth solution (elution solution) comprises 10 mM Tris-HCl, 0.1 mM EDTA, pH 8.0-9.0.

18. The method of claim 1, further comprising a step between steps d and e: flowing a washing solution-2 to wash contaminants of proteins, carbohydrates and small metabolites from the second layer.

19. The method of claim 17, wherein the washing solution-2 comprises 2-10 mM Tris-HCl, 80%-95% ethanol, pH 6-8.

20. A chromatography cassette or device for isolating nucleic acids, comprising:
a) a first solid layer of positively charged or cationic membrane or bead, and a second solid layer of membrane or bead which is coupled from the first layer by a coupling solution,
b) forwardly flowing a first solution (sample solution) containing a nucleic acid through the first layer to which the nucleic acid becomes bound, while solid particles in the first solution may block the first layer to reduce its flowability,
c) reversely flowing a second solution (the deblocking solution) through the first layer to which the nucleic acid still remains bound, and deblocking the first layer to increase its flowability,
wherein step b) and step c) alternate at least one time,
d) forwardly flowing a third solution (coupling solution) through the first layer from which the bound nucleic acid is eluted, and then flowing the second layer to which the eluted nucleic acid becomes bound, and
e) forwardly flowing a fourth solution (elution solution) through the second layer from which the bound nucleic acid is eluted,
whereby, the first layer can be deblocked to increase its flowability.


SEQUENCE LISTING (4 sequences)

SEQ ID NO: 1 - DNA, 30 bp, Homo sapiens
  modified_base 30: mod_base = OTHER; note = n is dideoxycytosine monophosphate
  source 1..30: mol_type = other DNA
tgggtataggggcgaaagactaatcgaacn

SEQ ID NO: 2 - DNA, 30 bp, Homo sapiens
  modified_base 30: mod_base = OTHER; note = n is dideoxycytosine monophosphate
  source 1..30: mol_type = other DNA
ctgagggaaacttcggagggaaccagctan

SEQ ID NO: 3 - DNA, 25 bp, Homo sapiens
  source 1..25: mol_type = other DNA
caaccgcgagaagatgacccagatc

SEQ ID NO: 4 - DNA, 25 bp, Homo sapiens
  source 1..25: mol_type = other DNA
gcaacgtacatggctggggtgttga